(19) United States
(12) Patent Application Publication
(10) Pub. No.: US 2005/0158265 A1
(43) Pub. Date: Jul. 21, 2005

(54) Cosmetic hair composition comprising tridecyl trimellitate and at least one fixing polymer

(76) Inventors: Sabine Vrignaud, Ia Plaine St. Denis (FR); Emmanuelle Belli, Asnieres (FR)

Correspondence Address: Thomas L. Irving;FINNEGAN, HENDERSON, FARABOW,, GARRETT & DUNNER, L.L.P., 1300 I Street, N.W., Washington, DC, 20005-3315, US

(21) Appl. No.: 10/866,864

(22) Filed: Jun. 15, 2004

Related U.S. Application Data
(60) Provisional application No. 60/505,167, filed on Sep. 24, 2003.

(30) Foreign Application Priority Data
Jun. 17, 2003 (FR) 03 07286

(51) Int. Cl. A61K 7/06; A61K 7/11

(52) U.S. Cl. 424/70.12; 424/70.15; 424/70.16

(57) ABSTRACT

A cosmetic hair composition comprising, in a cosmetically acceptable medium, tridecyl trimellitate and at least one fixing polymer; a cosmetic process, for example, a process for imparting at least one property to hair chosen from fixing a hairstyle and holding a hairstyle comprising applying the composition to hair, and also the use of the composition for imparting sheen to the hair.

This application claims benefit of U.S. Provisional Application No. 60/505,167, filed Sep. 24, 2003.

Disclosed herein are novel cosmetic hair compositions comprising tridecyl trimellitate and at least one fixing polymer. Also disclosed herein is a cosmetic process, for example, a process for imparting at least one property to hair chosen from fixing a hairstyle and holding a hairstyle using the compositions, and the use of the compositions for imparting sheen to hair.

The use of tribenzoic acid triester oils, such as tridecyl trimellitate, is well known for the formulation of many cosmetic products comprising a fatty phase.

This use is described, for example, in Patent Application No. EP-A-0792637 for preparing lipsticks. It is also mentioned in Patent Application No. EP-A-0194055 for preparing anhydrous cosmetic formulations without mineral oil. In U.S. Pat. No 4,940,577, this type of oil constitutes the fatty phase of transparent water-in-oil microemulsions. These microemulsions have a low water content and can be used as sunscreen products.

However, there is a need for hair products which may make it possible to impart to hair at least one of the following properties: providing hair with some hold and providing a long lasting appearance of sheen, without making it too markedly greasy in nature. These properties are not provided by compositions known in the art.

The present inventors propose to provide a solution to at least one of these problems seen in the prior art.

For example, the present inventors have discovered, surprisingly, that compositions comprising tridecyl trimellitate and at least one fixing polymer may give hair an intense and persistent sheen and, further, may result in hair that is particularly smooth to the touch.

It has also noted that these compositions adhere well to the hair and provide at least one good cosmetic property.

Finally, it has been discovered that these compositions can be formulated in an alcoholic medium.

Other subjects of the embodiments disclosed herein will emerge on reading the description and examples which follow.

Disclosed herein is thus a cosmetic hair composition comprising, in a cosmetically acceptable medium, tridecyl trimellitate and at least one fixing polymer.

Further disclosed herein is a cosmetic hair process for imparting at least one property to hair chosen from fixing a hairstyle and holding a hairstyle using the compositions.

Even further disclosed herein is the use of the compositions for imparting sheen to hair.

As used herein, “sheen” means a visual property of something that shines with reflected light; shininess, or luster.

Tridecyl Trimellitate

Tridecyl trimellitate is an oil of formula below:

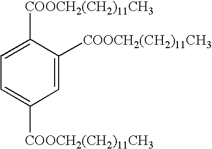

Examples of the tridecyl trimellitate include the commercial product sold under the name DUB TMTD by the company Stéarineries Dubois and the commercial product sold under the name LIPONATE TDTM by the company Lipo Chemicals.

The tridecyl trimellitate may be present in an amount ranging from 0.05 to 20% by weight, relative to the total weight of the composition, and, for example, ranging from 1 to 10% by weight, relative to the total weight of the composition.

Fixing Polymer

As used herein, the term “fixing polymer” means any polymer which makes it possible to give shape to hair or to maintain the shape of hair.

The at least one fixing polymer may, for example, be chosen from cationic, anionic, amphoteric, and nonionic fixing polymers.

Cationic Fixing Polvmers

The cationic fixing polymers which can be used in the compositions disclosed herein may, for example, be chosen from polymers comprising at least one group chosen from primary, secondary, tertiary and quaternary amine groups which are part of the polymer chain or directly connected thereto, and which have a number-average molecular mass ranging from 500 to 5 000 000, and, for example, ranging from 1000 to 3,000,000.

Examples of the cationic fixing polymers include the following:
(1) homopolymers or copolymers of acrylic or methacrylic esters or amides, comprising at least one amino functional group, wherein the homopolymers or copolymers comprise at least one of unit of formulae below: 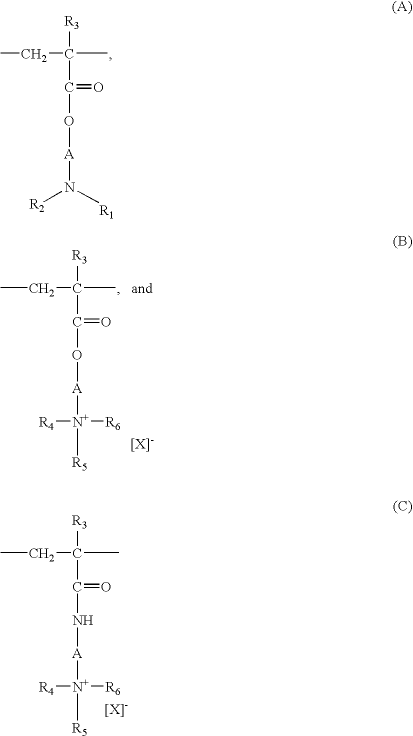
   wherein:
R₁ and R₂, which may be identical or different, are each chosen from a hydrogen atom and alkyl groups comprising from 1 to 6 carbon atoms;
R₃, which may be identical or different, is chosen from a hydrogen atom and a CH₃ group;
A is chosen from linear and branched alkyl groups comprising from 1 to 6 carbon atoms and hydroxyalkyl groups comprising from 1 to 4 carbon atoms;
R₄, R₅, and R₆, which may be identical or different, are each chosen from alkyl groups comprising from 1 to 18 carbon atoms and a benzyl group; and
X is chosen from a methosulphate anion and halides, such as chloride and bromide.

The copolymers of family (1) may also optionally comprise at least one unit derived from comonomers which can be chosen from acrylamides, methacrylamides, diacetone acrylamides, acrylamides, and methacrylamides substituted on the nitrogen with at least one group chosen from lower (C₁₋₄) alkyl groups, groups derived from acrylic or methacrylic acids and esters thereof, vinyllactams, such as vinylpyrrolidone and vinylcaprolactam, and vinyl esters.

Thus, examples of the copolymers of family (1) include:
copolymers of acrylamide and of dimethylaminoethyl methacrylate, quaternized with dimethyl sulphate or with a dimethyl halide, such as the product sold under the name HERCOFLOC® by the company Hercules,
copolymers of acrylamide and of methacryloyloxyethyltrimethylammonium chloride described, for example, in Patent Application No. EP-A-080976 and sold under the name BINA QUAT P 100 by the company Ciba Geigy,
copolymers of acrylamide and of methacryloyloxyethyltrimethylammonium methosulphate, such as the product sold under the name RETEN by the company Hercules,
quaternized or nonquaternized vinylpyrrolidone/dialkylaminoalkyl acrylate or methacrylate copolymers, such as the products sold under the name GAFQUAT by the company ISP, such as “Gafquat® 734” or “Gafquat® 755”, or the products called “Copolymer® 845, 958 and 937”. These polymers are described in detail in French Patent Nos. 2 077 143 and 2 393 573,
polymers comprising at least one fatty chain and at least one vinylpyrrolidone unit, such as the product sold under the name STYLEZE W20 and STYLEZE W10 by the company ISP,
dimethylaminoethyl methacrylate/vinylcaprolactam/vinylpyrrolidone terpolymers, such as the product sold under the name GAFFIX® VC 713 by the company ISP, and
quaternized vinylpyrrolidone/dimethylaminopropyl methacrylamide copolymers such as the product sold under the name GAFQUAT® HS 100 by the company ISP;
(2) cationic polysaccharides, for example, comprising quaternary ammonium, such as those described in U.S. Pat. Nos. 3,589,578 and 4,031,307, for example, guar gums comprising at least one trialkylammonium cationic group. Such products are, for example, sold under the trade names JAGUAR C13 S, JAGUAR C15, and JAGUAR C17 by the company Meyhall;
(3) quaternized copolymers of vinylpyrrolidone and vinylimidazole;
(4) chitosans or salts thereof; the salts which can be used may, for example, be chosen from acetate, lactate, glutamate, gluconate, and pyrrolidonecarboxylate of chitosan.

Examples of these chitosan compounds include the chitosan having a degree of deacetylation of 90.5% by weight, sold under the name Kytan Brut Standard by the company Aber Technologies and the chitosan pyrrolidonecarboxylate sold under the name KYTAMER® PC by the company Amerchol; and
(5) cationic cellulose derivatives such as the copolymers of cellulose or of cellulose derivatives grafted with a water-soluble quatemary ammonium monomer, and described, for example, in U.S. Pat. No. 4,131,576, such as hydroxyalkylcelluloses, for example, chosen from hydroxymethyl-, hydroxyethyl-, and hydroxypropyl celluloses grafted, for example, with at least one salt chosen from methacryloyloxyethyltrimethylammonium, methacrylamidopropyltrimethylammonium, and dimethyldiallylammonium salts.

The marketed products corresponding to this definition are, for example, the products sold under the names CELQUAT L 200 and CELQUAT H 100 by the company National Starch.

Anionic Fixing Polymers

The anionic fixing polymers may, for example, be chosen from polymers comprising at least one group derived from carboxylic, sulphonic, and phosphoric acid groups, and having a number-average molecular mass ranging from 500 to 5 000 000.

The carboxylic groups are provided by unsaturated monocarboxylic or dicarboxylic acid monomers such as those corresponding to the formula: 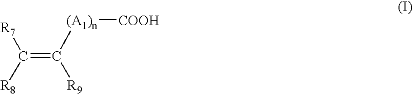
wherein:
n is an integer ranging from 0 to 10;
A₁ is a methylene group, optionally linked to the carbon atom of the unsaturated group or to the neighbouring methylene group when n is greater than 1, by a hetero atom, such as oxygen or sulphur;
R₇ is chosen from a hydrogen atom and phenyl and benzyl groups;
R₈ is chosen from a hydrogen atom and lower alkyl and carboxyl groups; and
R₉ is chosen from a hydrogen atom, lower alkyl groups, group —CH₂—COOH, and phenyl and benzyl groups.

In the abovementioned formula, the lower alkyl groups may, for example, be chosen from groups comprising from 1 to 4 carbon atoms, for example, methyl and ethyl groups.

In one embodiment, the anionic fixing polymers comprising at least one group derived from carboxylic groups may be chosen from:

A) homopolymers and copolymers of acrylic or methacrylic acid and salts thereof, such as the products sold under the names VERSICOL® E and K by the company Allied Colloid, and ULTRAHOLD® by the company BASF, copolymers of acrylic acid and of acrylamide sold in the form of their sodium salts, under the names RETEN 421, 423 and 425 by the company Hercules, and sodium salts of polyhydroxycarboxylic acids;

B) copolymers of acrylic or methacrylic acid with at least one monoethylenic monomer, for example, chosen from ethylene, styrene, vinyl esters, esters of acrylic, and esters of methacrylic acid, optionally grafted with at least one polyalkylene glycol, such as polyethylene glycol, and optionally crosslinked. Such polymers are described, for example, in French Patent No.1 222 944 and German Patent Application No. 2 330 956; the copolymers of this type comprise in their chain at least one unit chosen from optionally N-alkylated and hydroxyalkylated acrylamide units, as described, for example, in Luxembourg Patent Application Nos. 75370 and 75371, or proposed under the name QUADRAMER by the company American Cyanamid. Further examples include copolymers of acrylic acid and of C₁-C₄ alkyl methacrylate and terpolymers of vinylpyrrolidone, of acrylic acid and of C₁-C₂₀ alkyl, for example lauryl, methacrylate, such as the product sold by the company ISP under the name ACRYLIDONE® LM and methacrylic acid/ethyl acrylate/tert-butyl acrylate terpolymers such as the product sold under the name LUVIMER® 100 P by the company BASF.

Yet another example of the copolymers include the methacrylic acid/acrylic acid/ethyl acrylate/methyl methacrylate copolymers in aqueous dispersion, sold under the name AMERHOLD® DR 25 by the company Amerchol;

C) crotonic acid copolymers, such as those copolymers comprising in their chain at least one unit chosen from vinyl acetate and propionate units, and optionally at least one first additional unit, for example, chosen from allyl esters, methallyl esters, vinyl ethers and vinyl esters of linear or branched, saturated carboxylic acids comprising at least one long hydrocarbon-based chain, for example, chains comprising at least 5 carbon atoms, it being possible for these polymers to be optionally grafted or crosslinked, or, optionally, the crotonic acid copolymers may comprise at least one second additional unit chosen from vinyl, allyl, and methallyl esters of an α- or β-cyclic carboxylic acid. Such polymers are described, inter alia, in French Patent Nos. 1 222 944,1 580 545, 2 265 782, 2 265 781, 1 564 110 and 2 439 798. Commercial products which fall into this class are the resins 28-29-30, 26-13-14 and 28-13-10 sold by the company National Starch;

D) copolymers of monounsaturated C₄-C₈ carboxylic acids or anhydrides, chosen from:
copolymers comprising (i) at least one first unit chosen from maleic, fumaric, itaconic acids, and anhydrides and (ii) at least second unit chosen from vinyl esters, vinyl ethers, vinyl halides, phenylvinyl derivatives, acrylic acid and esters thereof, the anhydride functions of these polymers being optionally monoesterified or monoamidated. Such polymers are described, for example, in U.S. Pat. Nos. 2,047,398, 2,723,248 and 2,102,113, and GB Patent No. 839 805. Commercial products are, for example, those sold under the names GANTREZ® AN or ES by the company ISP; and
copolymers comprising (i) at least one first unit chosen from maleic, citraconic, and itaconic anhydride units and (ii) at least one second unit chosen from allyl and methallyl esters optionally comprising at least one group chosen from acrylamide, methacrylamide, α-olefin, acrylic ester, methacrylic ester, acrylic acid, methacrylic acid, and vinylpyrrolidone groups in their chain, wherein the anhydride functions of these polymers are optionally monoesterified or monoamidated.

These polymers are, for example, described in French Patent Nos. 2 350 384 and 2 357 241; and

E) polyacrylamides comprising at least one carboxylate group.

The homopolymers and copolymers comprising at least one group derived from sulphonic groups may be chosen from polymers comprising at least one unit chosen from vinylsulphonic, styrenesulphonic, naphthalenesulphonic, and acrylamidoalkylsulphonic units.

For example, these polymers may be chosen from:
salts of polyvinylsulphonic acid having a molecular mass ranging from 1000 to 100 000, and copolymers with an unsaturated comonomer, such as acrylic or methacrylic acids and esters thereof, and acrylamide and derivatives thereof, vinylethers, and vinylpyrrolidone;
salts of polystyrenesulphonic acid such as the sodium salts sold, for example, under the names FLEXAN® 500 and FLEXAN® 130 by National Starch. These compounds are described in Patent No. FR 2 198 719; and
salts of polyacrylamidesulphonic acids such as those mentioned in U.S. Pat. No. 4,128,631, and, for example, the polyacrylamidoethylpropanesulphonic acid sold under the name COSMEDIA POLYMER HSP 1180 by Henkel.

Another example of the anionic fixing polymers which can be used in the compositions disclosed herein include the anionic branched block polymer sold under the name FIXATE G100 by the company Noveon.

In one embodiment, the anionic fixing polymers may, for example, be chosen from the following:
acrylic acid copolymers, such as acrylic acid/ethyl acrylate/N-tert-butylacrylamide terpolymers sold, for example, under the name ULTRAHOLD® Strong by the company BASF,
copolymers derived from crotonic acid, such as the vinyl acetate/vinyl tert-butylbenzoate/crotonic acid terpolymers and the crotonic acid/vinyl acetate/vinyl neododecanoate terpolymers sold, for example, under the name RÉSINE 28-29-30 by the company National Starch,
polymers derived from maleic, fumaric or itaconic acids or anhydrides with vinyl esters, vinyl ethers, vinyl halides, phenylvinyl derivatives, acrylic acid and esters thereof, such as the methyl vinyl ether/monoesterified maleic anhydride copolymers sold, for example, under the name GANTREZ® by the company ISP,
copolymers of methacrylic acid and of methyl methacrylate sold under the name EUDRAGIT® L by the company Rohm Pharma,
copolymers of methacrylic acid and of ethyl acrylate sold under the name LUVIMER® MAEX or MAE by the company BASF,
vinyl acetate/crotonic acid copolymers sold, for example, under the name LUVISET CA 66 by the company BASF,
vinyl acetate/crotonic acid copolymers grafted with polyethylene glycol sold under the name ARISTOFLEX® A by the company BASF, and
polymers sold under the name FIXATE G100 by the company Noveon.

In yet another embodiment, the anionic fixing polymers may be chosen from the following:
monoesterified methyl vinyl ether/maleic anhydride copolymers sold under the name GANTREZ® ES 425 by the company ISP,
acrylic acid/ethyl acrylate/N-tert-butylacrylamide terpolymers sold under the name ULTRAHOLD® STRONG by the company BASF,
copolymers of methacrylic acid and of methyl methacrylate sold under the name EUDRAGIT® L by the company Rohm Pharma,
vinyl acetate/vinyl tert-butylbenzoate/crotonic acid terpolymers and crotonic acid/vinylacetate/vinyl neododecanoate terpolymers sold under the name RÉSINE 28-29-30 by the company National Starch,
copolymers of methacrylic acid and of ethyl acrylate sold under the name LUVIMER® MAEX and MAE by the company BASF,
vinylpyrrolidone/acrylic acid/lauryl methacrylate terpolymers sold under the name ACRYLIDONE® LM by the company ISP, and
polymers sold under the name FIXATE G100 by the company Noveon.
   Amphoteric Fixing Polymers

The amphoteric fixing polymers which can be used in the compositions disclosed herein can be chosen from polymers comprising units B and C distributed randomly in the polymer chain, wherein B is chosen from units derived from a monomer comprising at least one basic nitrogen atom and C is chosen from units derived from an acid monomer comprising at least one group chosen from carboxylic and sulphonic groups, or B and C, which can be identical or different, can be chosen from groups derived from zwitterionic monomers of carboxybetaines or sulphobetaines.

B and C, which can be identical or different, can also be chosen from cationic polymer chains comprising at least one group chosen from primary, secondary, tertiary, and quaternary amine groups, wherein at least one of the amine groups bears a carboxylic or sulphonic group linked via a hydrocarbon-based group, or B and C can form part of a chain of a polymer comprising ethylene-α,β-dicarboxylic units, wherein one of the carboxylic groups has been made to react with a polyamine comprising at least one group chosen from primary and secondary amine groups.

For example, the amphoteric fixing polymers may be chosen from the following polymers:
(1) copolymers comprising acidic vinyl units and basic vinyl units, such as those resulting from the copolymerization of a monomer derived from a vinyl compound bearing a carboxylic group, for example, acrylic acid, methacrylic acid, maleic acid, or alpha-chloroacrylic acid, and a basic monomer derived from a substituted vinyl compound comprising at least one basic atom, such as dialkylaminoalkyl methacrylate and acrylate and dialkylaminoalkylmethacrylamide and -acrylamide. Such compounds are described in U.S. Pat. No. 3,836,537;
(2) polymers comprising units derived from:
   a) at least one monomer chosen from acrylamides and methacrylamides substituted on the nitrogen atom with an alkyl group,
   b) at least one acidic comonomer comprising at least one reactive carboxylic group, and
   c) at least one basic comonomer, for example, chosen from acrylic and methacrylic acid esters comprising at least one substituent chosen from primary, secondary, tertiary and quaternary amine substituents, and the product of quaternization of dimethylaminoethyl methacrylate with dimethyl or diethyl sulphate.

The N-substituted acrylamides or methacrylamides may, for example, be chosen from compounds wherein the alkyl groups comprise from 2 to 12 carbon atoms, for example, N-ethylacrylamide, N-tert-butylacrylamide, N-tert-octylacrylamide, N-octylacrylamide, N-decylacrylamide, N-dodecylacrylamide, and corrresponding meth-acrylamides.

The acidic comonomers may, for example, be chosen from acrylic acid, methacrylic acid, crotonic acid, itaconic acid, maleic acid, fumaric acid, and alkyl monoesters, comprising from 1 to 4 carbon atoms, of maleic acids, fumaric acids, or anhydrides.

In one embodiment, the basic comonomers may be chosen from aminoethyl, butylaminoethyl, N,N′-dimethylaminoethyl, and N-tert-butylaminoethyl methacrylates.

The copolymers whose CTFA (4th edition, 1991) name is octylacrylamide/acrylates/butylaminoethyl methacrylate copolymer, such as the products sold under the name AMPHOMER® and LOVOCRYL® 47 by the company National Starch, may, for example, be used;
(3) crosslinked and acylated polyamino amides partially or totally derived from polyamino amides of general formula:
   CO—R₁₀—CO-Z (II)
   wherein:
R₁₀ is chosen from divalent groups derived from a saturated dicarboxylic acid, mono- or dicarboxylic aliphatic acids comprising at least one ethylenic double bond, esters of a lower alkanol comprising from 1 to 6 carbon atoms, of these acids, and groups derived from the addition of at least one of the acids to amines chosen from bis(primary) and bis-(secondary) amines and
Z is chosen from groups derived from a bis(primary), mono- or bis(secondary) polyalkylene-polyamines and may, for example, represent:
   a) in proportions ranging from 60 to 100 mol %, the group:
      —NH(CH₂)ₓ—NHₚ (IV)
      wherein x=2 and p=2 or 3, or x=3 and p=2,
this group being derived from a compound chosen from diethylenetriamine, triethylenetetraaminem and dipropylenetriamine;
   b) in proportions ranging from 0 to 40 mol %, the group (IV) above, wherein x=2 and p=1 and which is derived from a compound chosen from ethylenediamine and piperazine: 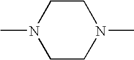
   c) in proportions ranging from 0 to 20 mol %, the group —NH—(CH₂)₆—NH— derived from hexamethylenediamine, these polyamino amides can be crosslinked by reaction addition of a difunctional crosslinking agent chosen from epihalohydrins, diepoxides, dianhydrides, and bis-unsaturated derivatives, using from 0.025 to 0.35 mol of crosslinking agent per amine group of the polyamino amide, and acylated by the action of acrylic acid, chloroacetic acid, an alkane sultone, or salts thereof.

In one embodiment, the saturated carboxylic acids may, for example, be chosen from acids comprising from 6 to 10 carbon atoms, such as adipic acid, 2,2,4-trimethyladipic acid, 2,4,4-trimethyladipic acid, terephthalic acid, and acids comprising at least one ethylenic double bond, such as acrylic acid, methacrylic acid, and itaconic acid.

The alkane sultones used in the acylation may, for example, be chosen from propane sultone and butane sultone, the salts of the acylating agents may, for example, be chosen from sodium and potassium salts;
(4) polymers comprising zwitterionic units of formula: 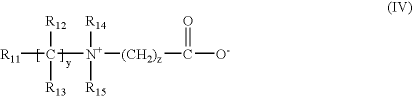
   wherein:
R₁₁ is chosen from polymerizable unsaturated groups, for example, acrylate, methacrylate, acrylamide, and methacrylamide groups;
y and z, which may be identical or different, are each integers ranging from 1 to 3;
R₁₂ and R₁₃, which may be identical or different, are each chosen from a hydrogen atom and methyl, ethyl, and propyl groups; and
R₁₄ and R₁₅, which may be identical or different, are each chosen from a hydrogen atom and alkyl groups chosen such that the sum of the carbon atoms in R₁₄ and R₁₅ does not exceed 10.

The polymers comprising such units can further comprise at least one additional unit derived from nonzwitterionic monomers, for example, chosen from dimethylaminoethyl acrylates, diethylaminoethyl acrylates, and methacrylates; alkyl acrylates and methacrylates; acrylamides and methacrylamides; and vinyl acetate.

By way of example, mention may be made of methyl methacrylate/methyl dimethylcarboxymethylammonioethylmethacrylate copolymers, such as the product sold under the name Diaformer Z301 by the company Sandoz;
(5) polymers derived from chitosan comprising monomer units corresponding to the following formulae: 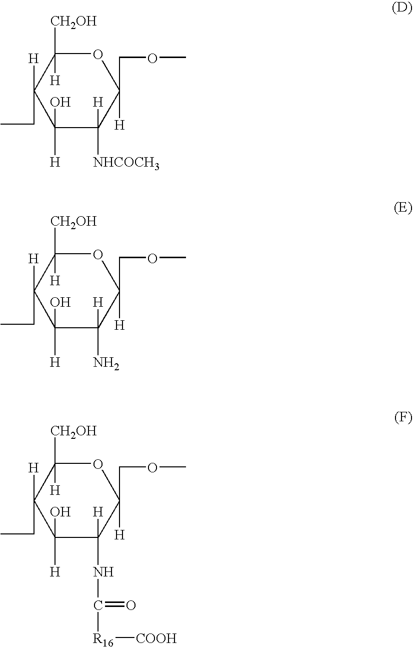
   wherein the unit (D) is present in proportions ranging from 0 to 30%, the unit (E) is present in proportions ranging from 5 to 50% and the unit (F) is present in proportions ranging from 30 to 90%, it being understood that, in the unit (F), R₁₆ is chosen from groups of formula: 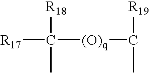
   wherein q=0 or 1,
if q=0, R₁₇, R₁₈ and R₁₉, which may be identical or different, are each chosen from a hydrogen atom; methyl, hydroxyl, acetoxy, and amino residues; monoalkylamine residues; and dialkylamine residues, optionally interrupted by at least one nitrogen atom and optionally substituted with at least one group chosen from amine, hydroxyl, carboxyl, alkylthio and sulphonic groups, and alkylthio residues wherein the alkyl group bears at least one amino residue, wherein at least one of the groups chosen from R₁₇, R₁₈ and R₁₉ are, in this case, a hydrogen atom; or
if q=1, R₁₇, R₁₈ and R₁₉, which may be identical or different, are each chosen from a hydrogen atom and salts formed by these compounds with bases or acids;
(6) polymers corresponding to formula (V) are, for example, described in French Patent No. 1 400 366: 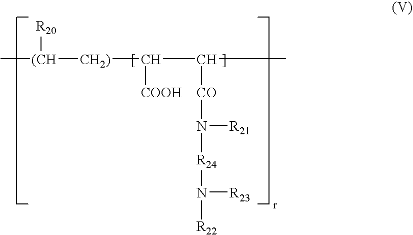
   wherein:
R₂₀ is chosen from a hydrogen atom, CH₃O, CH₃CH₂O, and phenyl groups;
R₂₁ is chosen from a hydrogen atom and lower alkyl groups, such as methyl and ethyl groups;
R₂₂ is chosen from a hydrogen atom and C₁-C₆ lower alkyl groups, such as methyl and ethyl groups;
R₂₃ is chosen from C₁-C₆ lower alkyl groups, such as methyl and ethyl groups, and groups corresponding to the formula: —R₂₄—N(R₂₂)₂, wherein R₂₄ is chosen from —CH₂—CH₂—, —CH₂—CH₂—CH₂—, and —CH₂—CH(CH₃)— groups and R₂₂ has the meanings mentioned above; and
r is an integer greater or equal to 1.
(7) polymers derived from the N-carboxyalkylation of chitosan, such as N-carboxymethylchitosan or N-carboxybutylchitosan, sold under the name “Evalsan” by the company Jan Dekker;
(8) amphoteric polymers of the type -D-X-D-X chosen from:
a) polymers obtained by the action of chloroacetic acid or sodium chloroacetate on compounds comprising at least one unit of formula:
   -D-X-D-X-D- (VI)
   wherein D is chosen from group 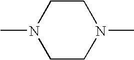
   and X is chosen from symbols E and E′, wherein E and E′, which may be identical or different, are each chosen from divalent alkylene groups comprising at least one chain chosen from straight and branched chains comprising up to 7 carbon atoms in the main chain, wherein the divalent alkylene radicals are optionally substituted with at least one hydroxyl group. E, and E′ can additionally comprise at least one entity chosen from oxygen, nitrogen, and sulphur atoms and 1 to 3 aromatic and heterocyclic rings; wherein the oxygen, nitrogen, and sulphur atoms may be present in the form of at least one group chosen from ether, thioether, sulphoxide, sulphone, sulphonium, alkylamine, alkenylamine, hydroxyl, benzylamine, amine oxide, quaternary amine, amide, imide, alcohol, ester, and urethane groups;
b) polymers of formula:
   -D-X-D-X- (VI′)
   wherein D is chosen from group: 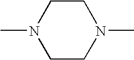
   and X is chosen from the symbols E and E′ and wherein at least one X is chosen from E′; E having the meaning given above and E′ is a divalent group chosen from divalent alkylene groups comprising at least one chain chosen from straight and branched chains comprising up to 7 carbon atoms in the main chain, wherein the divalent alkylene groups are optionally substituted with at least one hydroxyl group and comprising at least one nitrogen atom, wherein the nitrogen atom is substituted with an alkyl chain that is optionally interrupted by at least one oxygen atom, wherein the alkyl chain comprises at least one functional group chosen from carboxyl functional groups and hydroxyl functional groups, betainized by reaction with at least one reactant chosen from chloroacetic acid and sodium chloroacetate; and
(9) (C₁-C₅)alkyl vinyl ether/maleic anhydride copolymers partially modified by semiamidation with an N,N-dialkylaminoalkylamine such as N,N-dimethylamino-propylamine, or by semiesterification with an N,N-dialkylaminoalkanol. These copolymers can also comprise other vinyl comonomers such as vinylcaprolactam.

In one embodiment, the amphoteric fixing polymers described above are chosen from polymers of family (3), such as copolymers whose CTFA name is octylacrylamide/acrylates/butylaminoethyl methacrylate copolymer, such as the products sold under the names AMPHOMER®, AMPHOMER® LV 71 and LOVOCRYL® 47 by the company National Starch and those polymers of family (4), such as methyl methacrylate/methyl dimethylcarboxymethylammonioethylmethacrylate copolymers sold, for example, under the name DIAFORMER® Z301 by the company Sandoz.

Nonionic Fixing Polymers

The nonionic fixing polymers which can be used in the compositions disclosed herein may be chosen, for example, from:
polyalkyloxazolines;
vinyl acetate homopolymers;
vinyl acetate copolymers, such as copolymers of vinyl acetate and of acrylic ester, copolymers of vinyl acetate and of ethylene, and copolymers of vinyl acetate and of maleic ester, for example, of dibutyl maleate;
homopolymers and copolymers of acrylic esters, for example, copolymers of alkyl acrylates and of alkyl methacrylates, such as the products proposed by the company Rohm & Haas under the names PRIMAL® AC-261 K and EUDRAGIT® NE 30 D, by the company BASF under the name 8845, and by the company Hoechst under the name APPRETAN® N9212;
copolymers of acrylonitrile and of a nonionic monomer chosen, for example, from butadiene and alkyl(meth)acrylates; examples of such copolymers include the products provided under the name CJ 0601 B by the company Rohm & Haas;
styrene homopolymers;
styrene copolymers, for example, copolymers of styrene and of alkyl(meth)acrylate, such as the products MOWILITH® LDM 6911, MOWILITH® DM 611, and MOWILITH® LDM 6070 provided by the company Hoechst, the products RHODOPAS® SD 215 and RHODOPAS® DS 910 provided by the company Rhone Poulenc; copolymers of styrene, of alkyl methacrylate and of alkyl acrylate; copolymers of styrene and of butadiene; and copolymers of styrene, of butadiene, and of vinylpyridine;
polyamides;
vinyllactam homopolymers different from the vinylpyrrolidone homopolymers, such as the polyvinylcaprolactam sold under the name LUVISKOL® Plus by the company BASF; and
vinyllactam copolymers, such as a poly(vinylpyrrolidone/vinyllactam) copolymer sold under the trade name LUVITEC® VPC 55K65W by the company BASF; poly-(vinylpyrrolidone/vinyl acetate) copolymers, such as those sold under the name PVPVA® S630L by the company ISP and LUVISKOL® VA 73, VA 64, VA 55, VA 37 and VA 28 by the company BASF; and poly(vinylpyrrolidone/vinyl acetate/vinyl propionate)terpolymers such as the product sold under the name LUVISKOL® VAP 343 by the company BASF.

The alkyl groups in the nonionic polymers mentioned above may, for example, comprise from 1 to 6 carbon atoms.

In another embodiment, use may also be made of grafted silicone fixing polymers, comprising a polysiloxane portion and a portion comprising a nonsilicone organic chain, one of the two portions constituting the main chain of the polymer and the other portion is grafted onto the main chain.

These polymers are, for example, described in Patent Application Nos. EP-A-0 412 704, EP-A-0 412 707, EP-A-0 640 105, WO 95/00578, EP-A-0 582 152, WO 93/23009 and U.S. Pat. Nos. 4,693,935, 4,723,571, and 4,972,037.

The grafted fixing silicone polymers may be chosen from amphoteric, anionic, and non-ionic fixing polymers. In one embodiment, the grafted fixing silicone fixing polymers are chosen from anionic and nonionic fixing polymers.

Such polymers may, for example, be chosen from copolymers which can be obtained by free-radical polymerization using the mixture of monomers made up of:
a) from 50 to 90% by weight of tert-butyl acrylate,
b) from 0 to 40% by weight of acrylic acid,
c) from 5 to 40% by weight of a silicone macromer of formula 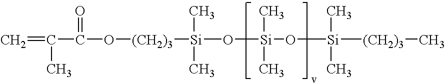
   wherein v is a number ranging from 5 to 700, the percentages by weight being calculated relative to the total weight of the monomers.

Other examples of grafted silicone polymers include polydimethylsiloxanes (PDMSs) onto which are grafted, via a thiopropylene-type connecting chain, mixed polymer units of poly((meth)acrylic acid) and of poly(alkyl(meth)acrylate), and polydimethylsiloxanes (PDMSs) onto which are grafted, via a thiopropylene connecting chain, isobutyl poly(meth)acrylate polymer units.

Further examples of grafted silicone fixing polymers include the product LUVIFLE® Silk sold by the company BASF.

The at least one fixing polymer may also be chosen from functionalized and nonfunctionalized, silicone and nonsilicone, cationic, nonionic, anionic, and amphoteric polyurethanes.

The polyurethanes may, for example, be chosen from those described in Patent Application Nos. EP 0 751 162, EP 0 637 600, EP 0 648 485, and FR 2 743 297, and in Patent Application Nos. EP 0 656 021 and WO 94/03510 from the company BASF, and EP 0 619 111 from the company National Starch.

Examples of polyurethanes which can used in the compositions disclosed herein include the products sold under the names LUVISET PUR® and LUVISET® SI PUR by the company BASF.

In the compositions disclosed herein, the at least one fixing polymer is present in an amount ranging from 0.01 to 20% by weight, for example, from 0.05 to 15% by weight, and, even further, for example, from 0.1 to 10%, by weight, relative to the total weight of the composition.

The cosmetically acceptable medium may, for example, comprise water; at least one cosmetically acceptable solvent, such as alcohols; or mixtures of water and at least one cosmetically acceptable solvent. The at least one cosmetically acceptable solvent may, for example, be chosen from C₁-C₄ alcohols.

Examples of alcohols that may be used as the at least one cosmetically acceptable solvent include ethanol and isopropanol. In one embodiment, ethanol may be used.

The compositions disclosed herein may further comprise at least one additive chosen from nonionic, anionic, cationic, amphoteric, and zwitterionic surfactants; fragrances; screening agents; preserving agents; proteins; vitamins; nonionic, anionic, cationic, amphoteric, and zwitterionic polymers other than the at least one fixing polymer described herein; mineral, plant, and synthetic oils; and agents and any other additives conventionally used in cosmetic compositions, such as anti-dandruff agents, anti-hairloss agents, dyes, pigments, moisturizers, such as glycerol and the like, polyols, and reducing agents.

The at least one additive may be present in an amount ranging from 0.001 to 20% by weight, relative to the total weight of the composition. The precise amount of each additive depends on its nature and is readily determined by those skilled in the art, and will depend on the chosen hair application.

Of course, those skilled in the art will take care to choose the additive(s) in such a way that the advantageous properties of the compositions disclosed herein are not, or are not substantially, altered by the envisaged addition(s).

The compositions disclosed herein can be used for producing many hair products, such as, products for fixing and/or holding the hair, conditioning products, or haircare products.

These compositions can be packaged in various forms. For example, the compositions can be provided in forms chosen from pump-dispenser bottles and aerosol containers, in order to apply the compositions in sprayed form or in mousse form. Such packaging forms may be used, for example, when it is desired to obtain a spray, a lacquer, or a mousse for fixing or treating the hair. The compositions disclosed herein can also be provided in forms chosen from creams, gels, emulsions, and lotions.

When the compositions disclosed herein are packaged in the form of an aerosol for the purpose of obtaining a lacquer or a mousse, it comprises at least one propellant which can be chosen from volatile hydrocarbons, such as n-butane, propane, isobutane, pentane, and halogenated hydrocarbons. The at least one propellant may also be chosen from carbon dioxide, nitrous oxide, dimethyl ether (DME), nitrogen, and compressed air. Mixtures of propellants may also be used. In one embodiment, dimethyl ether may be used.

For example, the at least one propellant may be present in an amount ranging from 5 to 90% by weight, and, further, for example, from 10 to 60% by weight, relative to the total weight of the composition.

Other than in the examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present invention. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the present disclosure are approximations, the numerical values set forth in the specific examples are reported as precisely as possible. Any numerical value, however, inherently contain certain errors necessarily resulting from the standard deviation found in their respective testing measurements. The following example is intended to illustrate the present disclosure without limiting the scope as a result.

### EXAMPLE

Composition A below was applied to 5 grams of a lock of natural euro-chestnut hair.

Composition A:

Composition A:

| | | |
|---|---|---|
| Liponate TDTM (1) | | 7% |
| Ultra Hold Strong (2) | | 5% |
| Ethanol | qs | 100% |

(1) tridecyl trimellitate sold by the company Lipochemicals
(2) anionic fixing polymer sold by the company BASF (acrylic acid/ethyl acrylate/N-tert-butylacrylamide terpolymer)

(1) tridecyl trimellitate sold by the company Lipochemicals
(2) anionic fixing polymer sold by the company BASF (acrylic acid/ethyl acrylate/N-tert-butylacrylamide terpolymer)

Composition A is applied to natural hair using a pump-dispenser bottle. After drying, it was observed that the lock had a sheen, was soft to the touch, and had a pleasant appearance.

1. A cosmetic hair composition comprising, in a cosmetically acceptable medium, tridecyl trimellitate and at least one fixing polymer.

2. The composition according to claim 1, wherein the tridecyl trimellitate is present in an amount ranging from 0.05 to 20% by weight, relative to the total weight of the composition.

3. The composition according to claim 2, wherein the tridecyl trimellitate is present in an amount ranging from 1 to 10% by weight, relative to the total weight of the composition.

4. The composition according to claim 1, wherein the at least one fixing polymer is chosen from cationic, anionic, amphoteric, and nonionic fixing polyurethanes.

5. The composition according to claim 4, wherein the cationic fixing polymers are chosen from homopolymers and copolymers of acrylic or methacrylic esters or amides comprising at least one amino functional group; cationic polysaccharides; quaternized copolymers of vinylpyrrolidone and vinylimidazole; and chitosans.

6. The composition according to claim 4, wherein the anionic fixing polymers are chosen from homopolymers and copolymers of acrylic or methacrylic acid and salts thereof, crotonic acid copolymers, copolymers of monounsaturated C₄-C₈ carboxylic acids or anhydrides, polyacrylamides comprising at least one carboxylate group, homopolymers and copolymers comprising at least one sulphonic group, anionic polyurethanes, and anionic grafted silicone polymers.

7. The composition according to claim 4, wherein the amphoteric fixing polymers are chosen from copolymers comprising acidic vinyl units and basic vinyl units; crosslinked and acylated polyamino amides; polymers comprising zwitterionic units, polymers derived from chitosan; partially modified (C₁-C₅)alkyl vinyl ether/maleic anhydride copolymers; amphoteric polyurethanes; and amphoteric grafted silicone polymers.

8. The composition according to claim 4, wherein the nonionic fixing polymers are chosen from polyalkyloxazolines, vinyl acetate homopolymers and copolymers, homopolymers and copolymers of acrylic esters, acrylonitrile copolymers, styrene homopolymers and copolymers, polyamides, vinyllactam homopolymers different from the vinylpyrrolidone homopolymers, vinyllactam copolymers, nonionic polyurethanes, and nonionic grafted silicone polymers.

9. The composition according to claim 4, wherein the at least one fixing polymer is present in an amount ranging from 0.01 to 20% by weight, relative to the total weight of the composition.

10. The composition according to claim 9, wherein the at least one fixing polymer is present in an amount ranging from 0.05 to 15% by weight, relative to the total weight of the composition.

11. The composition according to claim 10, wherein the at least one fixing polymer is present in an amount ranging from 0.1 to 10% by weight, relative to the total weight of the composition.

12. The composition according to claim 1, further comprising at least one additive chosen from nonionic, anionic, cationic, amphoteric and zwitterionic surfactants; fragrances; screening agents; preserving agents; proteins; vitamins; nonionic, anionic, cationic, and zwitterionic polymers other than the at least one fixing polymer; mineral, plant, and synthetic oils, anti-dandruff agents, anti-hairloss agents, dyes, pigments, moisturizers, and reducing agents.

13. The composition according to claim 1, wherein the composition is packaged in a form chosen from pump-dispenser bottles and aerosol containers.

14. The composition according to claim 1, wherein the composition is provided in a form chosen from creams, gels, emulsions, and lotions.

15. The composition according to claim 1, further comprising at least one propellant.

16. A method for cosmetic treatment of hair comprising applying to hair a composition comprising, in a cosmetically acceptable medium, tridecyl trimellitate and at least one fixing polymer.

17. A method for imparting at least one property to hair chosen from fixing a hairstyle and holding a hairstyle comprising applying to hair a composition comprising, in a cosmetically acceptable medium, tridecyl trimellitate and at least one fixing polymer.

18. A method for imparting sheen to hair comprising applying to hair a composition comprising, in a cosmetically acceptable medium, tridecyl trimellitate and at least one fixing polymer.
